(19) United States
(12) Patent Application Publication
(10) Pub. No.: US 2009/0234291 A1
(43) Pub. Date: Sep. 17, 2009

(54) CANNULA

(75) Inventors: Neil Saunders, South Jordan, UT (US); Eric Wayne Lindekugel, Fort Collins, CO (US); Brian Jacobs, West Jordan, UT (US); Duane Alan Ferkovich, Saratoga Springs, UT (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/388,429

(22) Filed: Feb. 18, 2009

Related U.S. Application Data
(60) Provisional application No. 61/030,533, filed on Feb. 21, 2008.

(51) Int. Cl. A61B 17/34

(52) U.S. Cl. 604/167.01

(57) ABSTRACT

A cannula assembly including a cannula body having a perfusion lumen extending therethrough; a removable introducer to be carried in the perfusion lumen, the removable introducer including a first hole, a second hole and a central lumen extending therebetween to allow fluid entering the first hole to flow through the central lumen and exit the second hole into the perfusion lumen; and a cap member including at least one venting means that allows air displaced by the fluid entering the first hole to be vented from the cannula body.

### CROSS-REFERENCE TO RELATED APPLICATION

This application claims the benefit of U.S. Provisional Application No. 61/030,533, filed Feb. 21, 2008, entitled “Improved Cannula”, which is also hereby incorporated herein by reference.

### BACKGROUND

1. Technical Field

The present invention relates generally to medical devices and methods, and more specifically to an improved cannula for aortic, femoral, and axillary use that may use an introducer for vascular insertion.

2. Related Art

The cannula of choice for most applications is femoral cannula that is flexible and uses an introducer to provide structural support to facilitate insertion. Current market trends have identified that surgeons are now using femoral cannula for aortic applications. The reason for this trend is that patients are expecting smaller incisions resulting from medical procedures which translate to faster recovery times and less scarring. The femoral cannula with an introducer fits the need for small incision procedures.

Unfortunately, femoral cannula designs do not provide automatic air venting capabilities, and most do not have satisfactory cannula to introducer edge transitions to facilitate smooth insertions into diseased aortas. For example, currently surgeons who use femoral cannula for aortic applications are forced to accept cannula with gaps between the introducer outer diameter (OD) and the cannula inner diameter (ID), which creates a larger step transition that may “catch” tissue and possibly tear a fragile diseased aorta (See FIG. 6). Also, a surgeon must manually vent air from the cannula before the surgeon may push the surgeon may release air emboli into the aorta increasing the risk for a patient stroke. Moreover, the manual venting releases excessive amounts of blood within the sterile field and may spray clinicians within the area.

### SUMMARY

The present invention is directed to an improved cannula with an introducer in which a structure for automatic venting of the cannula is embodied in the length of the introducer and a cap of the cannula body. Moreover, the automatic venting allows the cannula to introducer transition edge to have a reduced profile to facilitate smooth insertions into diseased aortas.

The introducer is formed to incorporate an opening or hole that allows blood to fill the cannula body via the introducer tip opening or hole, which is typically designed for tracking guidewires within the vasculature. A self venting cap is provided which allows for a semi-seal between the introducer and the cannula body, while allowing air to escape when being displaced by blood filling the cannula body, thereby protecting the clinician and minimizing blood loses while in use.

Further, the self venting cap allows the cannula to be designed with a minimal cannula to introducer transition edge, using, for example, a tight or “interference” fit, with virtually no gap, between the introducer OD and the cannula ID to minimize the height of the cannula tip stepped edge.

In one aspect, a cannula assembly is provided including a cannula body having a perfusion lumen extending therethrough. A removable introducer, including a tip hole, a side hole and a central lumen extending therebetween, is carried in the perfusion lumen. The introducer allows fluid entering the tip hole to flow through the central lumen and exit a side hole into the perfusion lumen. A cap member, including at least one vent channel, allows air displaced by the blood entering the tip hole of the introducer to be vented from the cannulae body.

The foregoing and other features and advantages of the invention will become more apparent from the following detailed description, which proceeds with reference to the accompanying figures.

### BRIEF DESCRIPTION OF THE DRAWINGS

The features, objects, and advantages of the invention will become more apparent from the detailed description set forth below when taken in conjunction with the drawings, wherein:

FIG. 1 is a side view of a cannula assembly in accordance with an embodiment of the present invention;

FIG. 2A is a simplified side view of a portion of the cannula assembly of FIG. 1 in accordance with an embodiment of the present invention;

FIG. 2B is a simplified side view of a portion of the introducer of FIG. 2A in accordance with an embodiment of the present invention;

FIG. 2C is a view of the tip of the cannula assembly in accordance with an embodiment of the present invention;

FIGS. 3A and 3B are axial and perspective views, respectfully, of the cap member in accordance with an embodiment of the present invention;

FIG. 4 is a simplified view of the use of the cannula assembly in a human aorta in accordance with an embodiment of the present invention;

FIG. 5 is a simplified view of the cannula assembly, shown in partial section, in accordance with an embodiment of the present invention;

FIG. 6 is a tip view of a typical cannula with an introducer;

FIGS. 7A and 7B are simplified illustrations of another embodiment of the cannula assembly;

FIGS. 8A and 8B are simplified illustrations of another embodiment of the cannula assembly;

FIGS. 9A and 9B are simplified illustrations of another embodiment of the cannula assembly; and

FIGS. 10A-10H are simplified illustrations of various embodiments of the cap member and means for venting the cap member in accordance with the present invention.

### DETAILED DESCRIPTION

The following description is exemplary in nature and is not intended to limit the scope, applicability, or configuration of the invention in any way. Various changes to the described embodiments may be made in the function and arrangement of the elements described herein without departing from the scope of the invention.

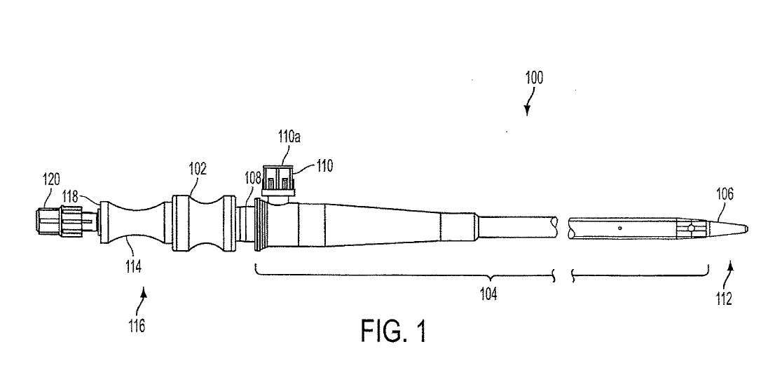

FIG. 1 is a side view of a cannula assembly 100 in accordance with an embodiment of the present invention. Cannula assembly 100 includes a cap member 102 disposed at a distal end of cannula assembly 100, and a cannula body 104 concentrically surrounding an introducer 106.

Introducer 106 may have a blunt distal end 112 and a handle 114 at a proximal end 116 of cannula assembly 100 to abut cap member 102 as introducer 106 moves into the end of cap member 102 and into cannula assembly 100 in a slip-lock relationship. Introducer 106 may also include a cap 120 that is used to close-off introducer 106, for example, when a guidewire is not being used to track introducer 106 within the vasculature. Cap 120 may include a porous cap or plug to ensure that the central lumen of introducer 106 may vent air and seal when blood comes in contact with the cap.

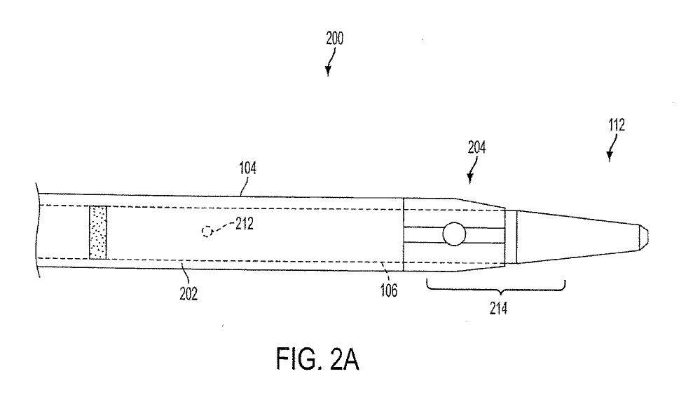

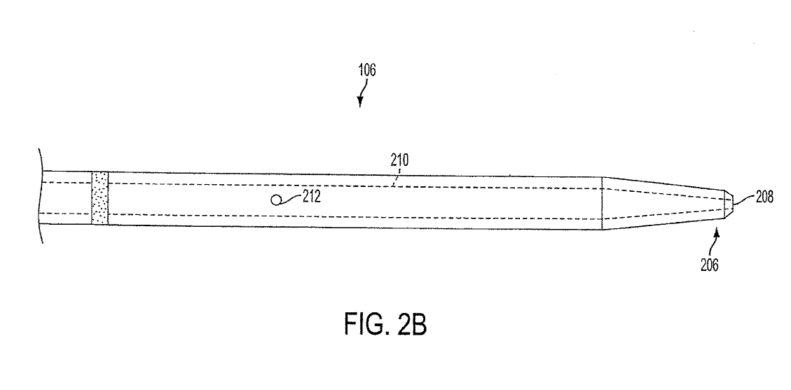

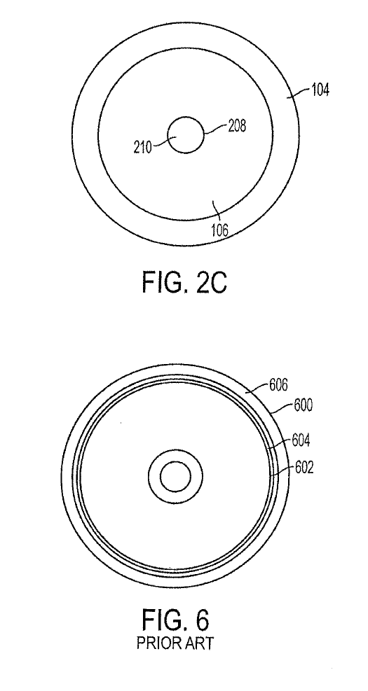

FIG. 2A illustrates a distal portion 200 of cannula assembly 100 that includes a portion of cannula body 104 and a portion of introducer 106 as part of the assembly. In one embodiment, cannula body 104 includes a central perfusion lumen 202, which extends from a barbed proximal end 108 (FIG. 1) to a distal end of cannula body 104, also referred to as cannula tip 204. Introducer 106 may be removably received via cap member 102 into perfusion lumen 202 to facilitate the introduction of cannula assembly 100 into the body vasculature as will be later described. Cannula body 104 may be formed of any suitable flexible plastic material and may include a reinforcing coiled spring (not shown) embedded in the wall of cannula body 104.

As further illustrated in FIGS. 2B and 2C, introducer 106 may be formed as a tube having a tip hole 208 defined at the distal end 206 that serves as entry into a central lumen 210 defined axially through introducer 106.

In one embodiment, introducer 106 further defines at least one opening or hole 212 (hereinafter “hole 212”), to a plurality of holes 212, defined through the wall of introducer 106 to be in communication with central lumen 210. Hole 212 may be positioned anywhere along the length of introducer 106 between the distal tip of cannula 204 and proximal end of cannula assembly 100.

In this embodiment, hole 212 of introducer 106 allows fluid, typically blood, entering introducer 106 through tip hole 208 to fill central lumen 210. In one embodiment, blood fills central lumen 210 until it reaches hole 212. The blood is then able to exit hole 212 and fill or prime perfusion lumen 202 of cannula body 104 which surrounds introducer 106.

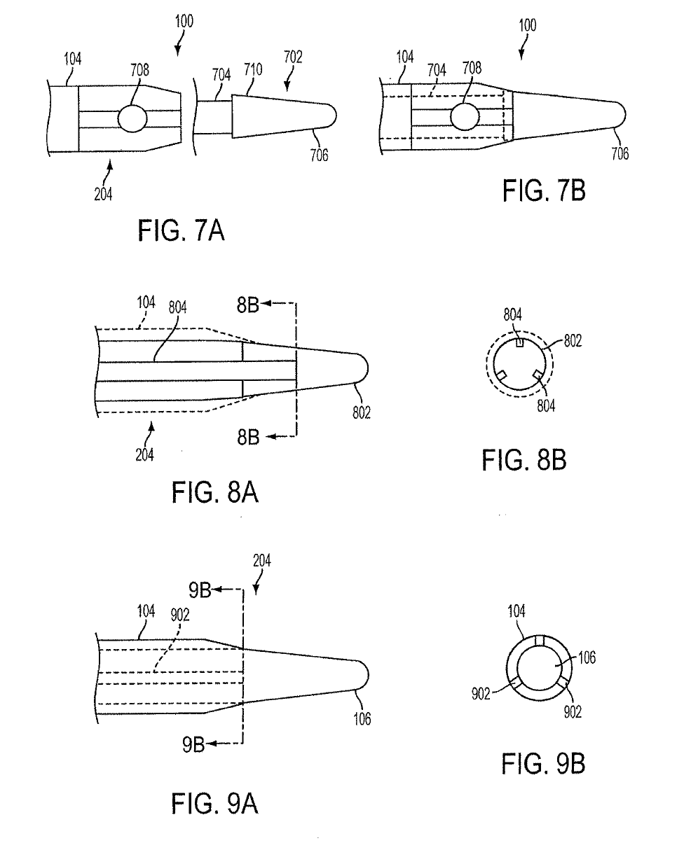

FIGS. 7A and 7B illustrate another embodiment of cannula assembly 100 that allows a fluid, such as blood, to enter perfusion lumen 202. In this embodiment, introducer 702 includes an introducer tip 706 having a proximal end portion 710 that is sized to engage a distal end of cannula body 104 as described above. Introducer 702 also includes a portion 704 having a reduced OD relative to proximal end portion 710 of introducer tip 706. In this embodiment, distal tip 204 of cannula body 104 includes at least one opening 708 defined thereon to be in communication with perfusion lumen 202. Opening 708 is positioned to be adjacent to portion 704 when introducer 702 is assembled for deployment. Because of the reduced OD of portion 704, blood is allowed to enter into opening 708 and fill perfusion lumen 202.

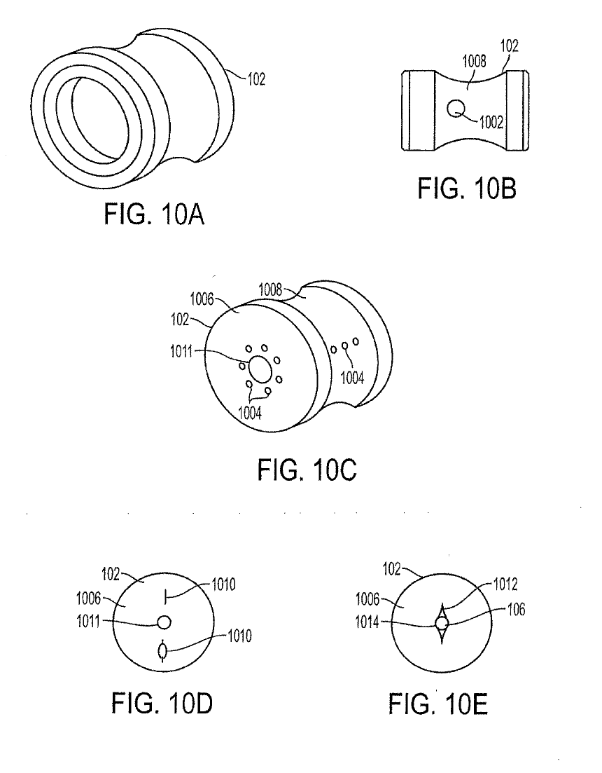

FIGS. 8A and 8B illustrate yet another embodiment of cannula assembly 100 that allows a fluid, such as blood, to enter perfusion lumen 202. In this embodiment, introducer 802 includes at least one channel or groove 804 formed or cut axially along the OD of introducer 802. Groove 804 may extend from a position within perfusion lumen 202 to a position distal to distal cannula tip 204 of cannula body 104 (shown dashed). Blood contacting introducer 802 may enter perfusion lumen 202 through groove 804.

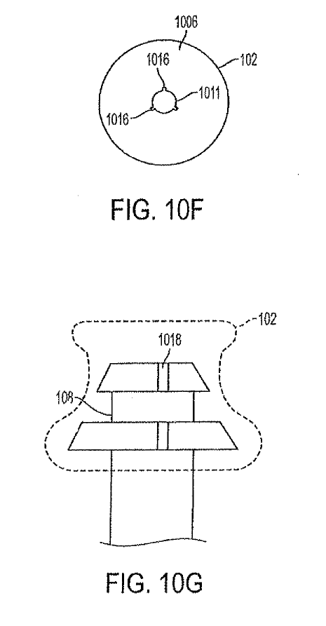

FIGS. 9A and 9B illustrate yet another embodiment of cannula assembly 100 that allows a fluid, such as blood, to enter perfusion lumen 202. In this embodiment, cannula body 104 includes at least one channel or groove 902 formed or cut axially along the ID of cannula body 104. Groove 902 may extend from a position within perfusion lumen 202 to the extent of distal cannula tip 204 of cannula body 104. Blood contacting cannula tip 204 may enter perfusion lumen 202 through groove 902.

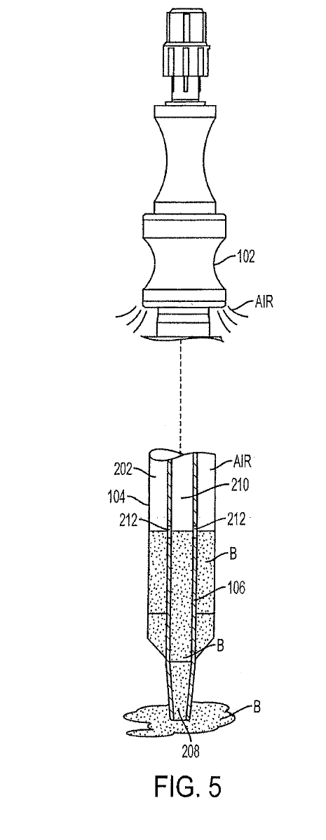

Referring now to FIG. 6, a cannula body 600 with an introducer 602 is shown having a gap 604 defined between the OD of introducer 602 and the ID of cannula body 600. In this device, gap 604 is provided to allow blood to enter cannula body 600. However, gap 604 may create a step edge 606, in some instances having a height of approximately 0.030 inches that may catch and damage tissue during insertion. Beneficially, in the present invention, blood is allowed to fill perfusion lumen 202 of cannula body 104 via, for example, the embodiments shown, for example, in FIGS. 2B-2C, 7A-7B, 8A-8B and 9A-9B that do not create a substantial step edge.

Instead, a tip transition 214 (FIG. 2A), defined as the point at which introducer 106 exits distal cannula tip 204 of catheter body 104, may be made with a tapered down edge on cannula tip 204, to provide substantially an “interference fit.” The interference fit allows tip transition 214 to be made with an outer edge profile from about 0.000 to about 0.005 inches. The low profile, tip transition 214 translates to safer insertion of cannula body 104 into diseased arteries.

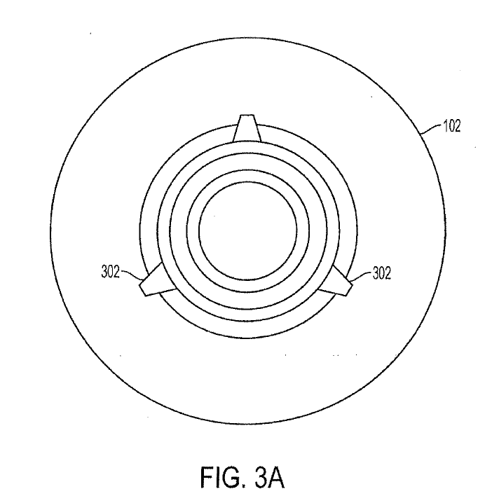

FIGS. 3A and 3B are axial and perspective views of cap member 102 shown as part of cannula assembly in FIG. 1. Cap member 102 provides a seal between introducer 106 and cannula body 104, such that a fluid entering cannula body 104 may be substantially maintained within cannula body 104 and perfusion lumen 202.

In one embodiment, cap member 102 includes channel vents 302 formed within the inner surface 304 of cap member 102. As explained below, channel vents 302 provide a means for allowing air to escape from within cannula body 104.

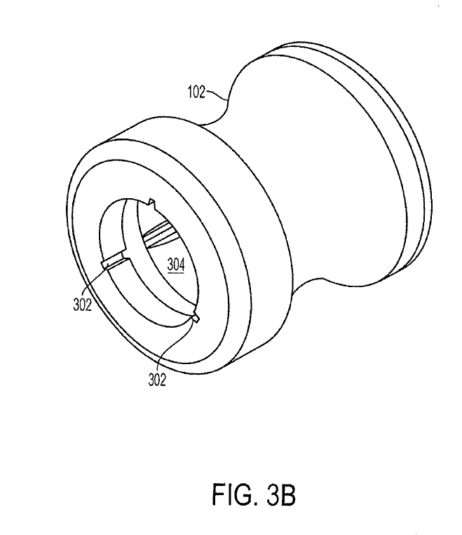

In an operational embodiment, as shown in FIG. 4, cannula assembly 100 including introducer 106 is introduced into the aorta. In this embodiment, introducer 106 is inserted in a slip-lock relationship through cap member 102 into perfusion lumen 202 of cannula body 104. Introducer 106 is moved down through perfusion lumen 202 so that distal end 206 exits the distal end of cannula body 104 (tip transition 214, FIG. 2A) to create a “tip” on the end of cannula body 104 to facilitate its entry into the aorta.

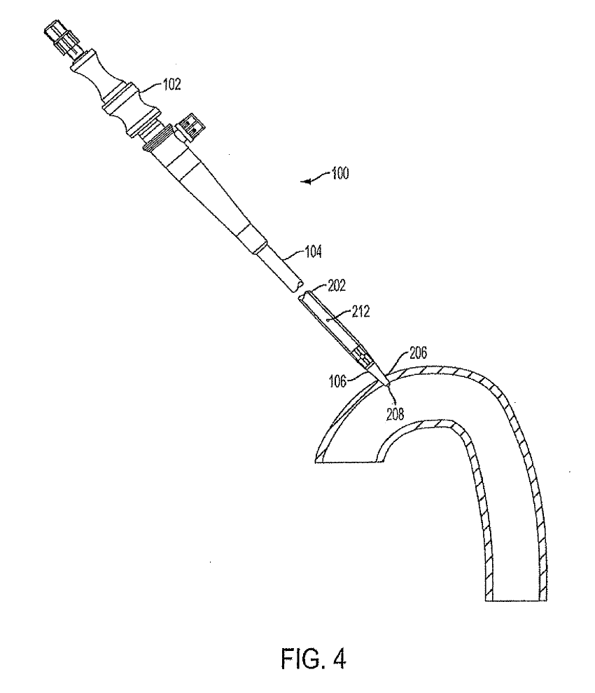

Referring now to FIGS. 2A-2C, 3A, 3B, 4 and 5, in one embodiment, as introducer 106 enters the aorta, blood B enters tip hole 208 and begins to fill central lumen 210. The blood B continues to fill central lumen 210 until it reaches hole 212. Blood B may then spill from hole 212 into perfusion lumen 202, which causes perfusion lumen 202 to fill with blood B.

However, in order for blood B to fill perfusion lumen 202, air that is present inside perfusion lumen 202 needs to be vented. Otherwise, arterial pressure compresses and traps the air and the cannula body does not fill, usually requiring a surgeon to manually break a seal between the cannula body and the environment.

Thus, in the present invention, air being displaced by blood B entering perfusion lumen 202 is pushed through perfusion lumen 202 until it reaches cap member 102. As displaced air reaches the proximal end of cannula body 104, the air is pushed out from barbed end 108 disposed within cap member 102 and allowed to vent from cap member 102 through channel vents 302. Channel vents 302 allow blood B to escape perfusion lumen 202 and cannula body 104 as well, with minimal blood loss. Placement of channel vents 302 on the distal end of cap member 102 directs any leaking or weeping blood down and away from the clinicians at the proximal end.

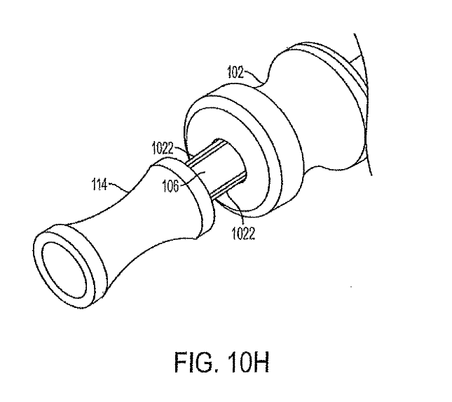

As shown in FIGS. 10A-H, alternative means may be used to allow air to vent from within cap member 102 to the environment. As shown in FIGS. 10A and 10B, cap member 102 may be formed or manufactured entirely of a porous material that allows air to permeate through the entire cap member 102. Alternatively, only a portion of cap member may be made of the porous material or, a separately manufactured porous insert 1002 may be inserted into cap member 102. Porous materials may include, for example, sintered plastic resin, fabric or filter membranes.

As shown in FIG. 10C, cap member 102 may be formed or manufactured having one to a plurality of pin holes 1004 that allow air to vent from within cap member 102. In various embodiments, pin holes 1004 may be located on the top proximal surface 1006 of cap member 102 or along the axial surface 1008.

As shown in FIGS. 10D and 10F, one to a plurality of slits 1010 may be cut into proximal surface 1006 that allow air to vent from within cap member 102. In one embodiment, slits 1010 may be made to open wider by applying manual pressure to cap member 102, such as by “squeezing” cap member 102. In another embodiment, a slit 1012 may be used in place of the proximal opening 1011 of the lumen which receives introducer 106 into cap member 102. Since slit 1012 is longer than the diameter of introducer 106, spaces 1014 are created at the ends of slit 1012 that allow air to vent from cap member 102.

As shown in FIG. 10F, opening 1011 of the lumen to receive introducer 106 into cap member 102 may include channels or grooves 1016 formed thereon to allow air to escape from within cap member 102.

As shown in FIG. 10G, barbed end 108 of cannula body 104 may have channels or grooves 1018 formed or cut into the barbs to allow air to vent from cap member 102.

As shown in 10H, introducer 106 may include one to a plurality of channels or grooves 1022 formed or cut into the proximal end of introducer 106 at a point just distal and adjacent to handle 114. Grooves 1022 may extend into cap member 102 a distance that is appropriately determined to allow air to escape from within cap member 102 via grooves 1022.

Referring again to FIG. 1, in one embodiment, T-port connector 110 may include a porous material or valved assembly within cap 110a at the end of connector 110 that allows air to vent from perfusion lumen 202 in accordance with an alternative embodiment of the present invention.

The invention has been disclosed in an illustrative manner. Accordingly, the terminology employed throughout should be read in an exemplary rather than a limiting manner. Although minor modifications of the invention will occur to those of ordinary skill in the art, it shall be understood that what is intended to be circumscribed within the scope of the patent warranted hereon are all such embodiments that reasonably fall within the scope of the advancement to the art hereby contributed, and that scope shall not be restricted, except in light of the appended claims and their equivalents.

What is claimed is:

1. A cannula assembly comprising:
a cannula body having a perfusion lumen extending therethrough;
a removable introducer to be carried in the perfusion lumen, the removable introducer including a first hole, a second hole and a central lumen extending therebetween to allow fluid entering the first hole to flow through the central lumen and exit the second hole into the perfusion lumen; and
a cap member including a venting means that allows air displaced by the fluid entering the first hole to be vented.

2. The cannula assembly of claim 1, wherein the fluid comprises blood.

3. The cannula assembly of claim 1, wherein said first hole is located at a tip of the removable introducer and the second hole is located on an axial wall of the removable introducer.

4. The cannula assembly of claim 1, wherein said venting means comprises grooves formed within said cap member that channel air out of said cap member.

5. The cannula assembly of claim 1, wherein said venting means comprises at least a portion of the cap member comprising a porous material.

6. The cannula assembly of claim 1, wherein said venting means comprises said cap member including at least one pin hole thereon.

7. The cannula assembly of claim 1, wherein said venting means comprises said cap member including at least one slit thereon.

8. The cannula assembly of claim 1, wherein a transition between the cannula body and the removable introducer comprises an edge having a height no greater than about 0.005 inches.

9. A cannula assembly comprising:
a cannula body having a perfusion lumen extending therethrough, and an introducer disposed in the perfusion lumen, the cannula body, introducer and perfusion lumen including a means for allowing a fluid to flow into said perfusion lumen; and
a venting means for allowing air displaced by the fluid entering the perfusion lumen to be vented from the cannula body.

10. The cannula assembly of claim 9, wherein the fluid comprises blood.

11. The cannula assembly of claim 9, wherein said means for allowing a fluid to flow into said perfusion lumen comprises the introducer including a first hole, a second hole and a central lumen extending therebetween to allow fluid entering the first hole to flow through the central lumen and exit the second hole into the perfusion lumen.

12. The cannula assembly of claim 9, wherein said means for allowing a fluid to flow into said perfusion lumen comprises the introducer including at least one groove formed on said introducer which channels the fluid into the perfusion lumen.

13. The cannula assembly of claim 9, wherein said means for allowing a fluid to flow into said perfusion lumen comprises the cannula body defining a first hole, the introducer having a reduced diameter portion adjacent to the first hole when said introducer is disposed in said perfusion lumen.

14. The cannula assembly of claim 9, wherein the venting means comprises at least one groove formed on a cap member.

15. The cannula assembly of claim 9, wherein the venting means comprises at least one pin hole formed on a cap member.

16. The cannula assembly of claim 9, wherein a transition between the cannula body and the introducer comprises an edge having a height of no greater than about 0.005 inches.

17. The cannula assembly of claim 9, wherein a transition between the cannula body and the introducer includes no gap between an internal diameter of the cannula body and an outer diameter of the introducer.

18. A cannula assembly comprising:
a cannula body having a perfusion lumen extending therethrough;
a removable introducer disposed in the perfusion lumen, the removable introducer including at least one opening defined on the removable introducer between a proximal end and a distal end of the cannula body in communication with a central lumen extending through the removable introducer and a tip hole that allows blood to enter the central lumen from the distal end of the introducer, the at least one opening allowing the blood to overflow from the central lumen and into the perfusion lumen; and
a cap member configured to allow air displaced by the blood entering into the central lumen to be automatically vented from the cannula body, a transition between the cannula body and the removable introducer includes no gap between an internal diameter of the cannula body and an outer diameter of the removable introducer.

19. The cannula assembly of claim 18, wherein the cap member comprises at least one opening that allows environmental communication between an inside of the cap member and the external environment.

20. The cannula assembly of claim 18, wherein the cannula body and the removable introducer have an interference fit at the distal end of the cannula body.
